(19) United States
(12) Patent Application Publication
(10) Pub. No.: US 2026/0250368 A1
(43) Pub. Date: Aug. 27, 2026

(54) SUPPRESSION OF UVEITIS BY SINGLE DOMAIN ANTIBODY

(71) Applicants: Singh Biotechnology, LLC, Clearwater, FL (US); The U.S.A., as represented by the Secretary, Department of Health and Human Services, Bethesda, MD (US)

(72) Inventors: Sunanda Singh, Clearwater, FL (US); Charles E. Egwuagu, Bethesda, MD (US)

(73) Assignees: Singh Biotechnology, LLC, Clearwater, FL (US); The U.S.A., as represented by the Secretary, Department of Health and Human Services, Bethesda, MD (US)

(21) Appl. No.: 19/662,470

(22) Filed: Apr. 29, 2026

Related U.S. Application Data
(63) Continuation of application No. 17/859,861, filed on Dec. 21, 2022.
(60) Provisional application No. 63/219,161, filed on Jul. 7, 2021.

(51) Int. Cl. C07K 16/18; A61P 27/02; A61P 29/00

(52) U.S. Cl. CPC C07K 16/18; A61P 27/02; A61P 29/00; C07K 2317/569

(57) ABSTRACT

Methods and compositions for treating uveitis are provided. The methods include administering to a subject a single-domain antibody (sdAb) comprising the amino acid sequence set forth in SEQ ID NO:1. In certain embodiments, the sdAb is administered systemically at a therapeutically effective dose and inhibits STAT3 activation in CD4+ T-cells, thereby reducing Th17-mediated inflammation. The methods may be used to treat autoimmune uveitis and may improve retinal function as measured by electroretinogram (ERG) amplitude. Also provided are pharmaceutical compositions comprising the sdAb and a pharmaceutically acceptable carrier. Further methods of modulating immune responses associated with uveitis are disclosed, including reducing cytokine production and T-cell-mediated inflammation.

### CROSS-REFERENCE TO RELATED APPLICATION

This application is a continuation of U.S. patent application Ser. No. 17/859,861, filed Dec. 21, 2022, which claims the benefit of U.S. Provisional Patent Application No. 63/219,161, filed on Jul. 7, 2021 the contents of which are incorporated herein by reference.

### FEDERAL FUNDING NOTICE

This invention was made with Government Support under project number Z01 #: EY000315-29 by the National Institutes of Health, National Eye Institute. The United States Government has certain rights in the invention.

### SEQUENCE LISTING

The present application is being filed along with a Sequence Listing in electronic format. The Sequence Listing is provided as a file titled “Sequence Listing XML” created Jul. 5, 2022, and is 4,016 bytes in size. The information in the electronic format of the Sequence Listing is incorporated herein by reference in its entirety.

### BACKGROUND

Cytokines such as IFN-7, IL-2, IL-4, IL-6, IL-10, IL-21, IL-23, IL-27, and IL-35 that regulate immune responses and autoimmune diseases mediate their biological activities through the activation of the Janus Kinase (JAK)/STAT pathway. This evolutionary conserved signal transduction pathway is orchestrated by the four Janus Kinases (Jak1, Jak2, Jak3, Tyk2) and the 7-member signal transducer and activator of transcription factor (STAT) family of proteins, STAT1, STAT2, STAT3, STAT4, STAT5a, STAT5b, and STAT6. Binding of a cytokine to its cognate receptor activates the requisite Jak proteins by transphosphorylation, providing docking sites for recruitment of specific STATs. STATs recruited to the receptor complex are phosphorylated at a critical tyrosine residue, form homo- or hetero-dimers and translocate into the nucleus where they bind to specific DNA sequences and activate gene transcription. Thus, the JAK/STAT pathway provides a rapid membrane to nucleus mechanism that transduces signals from the cell membrane to the nucleus and couples specific gene expression to change in the behavior of the cell.

STAT3 is unique among STAT proteins because it plays an essential and non-redundant role in mammalian cells. In mice, genetic deletion of stat3 results in embryonic lethality and death within 3 weeks after birth. Dominant-negative mutations in the DNA-binding domain of STAT3 is the cause of the rare immunodeficiency disorder known as Job's syndrome, for which there is no cure. Although much is known about the role of aberrant activation of STAT3 which results in uncontrolled proliferation, cell growth and oncogenesis, STAT3 has wide-ranging functions in T-cells and serves as a convergence point for mechanisms that regulate lymphocyte quiescence and those controlling T-cell activation and survival. In contrast to its role in promoting proliferation of activated T-cells, it maintains T-cells at the G0 phase of the cell cycle by binding the FoxO1 or FoxO3a promoter and upregulating the expression of these Class-O Forkhead transcription factors which play essential roles in maintaining T-cells in quiescent state. Furthermore, STAT3-deficiency in T-cells results in downregulation of FoxO1, FoxO3a and marked decrease of FoxO-target genes such as IkB and p27Kip1, leading to enhancement of NF-kB activation and production of IL-2. On the other hand, STAT3 is required for activation of Th17 master transcription factor, RORγt transcription and the expression of its signature proinflammatory cytokine IL-17. Importantly, mice with targeted deletion in CD4+ T-cells are resistant to development of experimental autoimmune uveitis (EAU) and experimental autoimmune encephalomyelitis, indicating that STAT3 is a potential therapeutic target for these central nervous system (CNS) autoimmune diseases and other autoinflammatory diseases.

STAT3 is not easily targeted pharmacologically because it is an intracellular protein. Several noninvasive methods have been used to deliver STAT3 mimetic peptides coupled to membrane permeable hydrophobic lipohilic motifs to specifically inhibit STAT3 SH2 domains or binding of STAT3 to kinase inhibitory sites on JAK or cytokine receptors with varying degrees of success.

The present invention relates to the use of single-domain antibodies (sdAbs), proteins and polypeptides directed against STAT3 intracellular components that cause a condition or disease such as uveitis. The invention also includes nucleic acids encoding the sdAbs, proteins and polypeptides, and compositions comprising the sdAbs. The invention includes the use of the compositions, sdAbs, proteins or polypeptides for prophylactic, therapeutic or diagnostic purposes. The invention also includes the use of monoclonal antibodies directed towards the sdAbs of the invention.

### SUMMARY

The present disclosure relates to methods and compositions for treating uveitis and modulating immune responses associated therewith. In particular, the disclosure provides methods of treating uveitis in a subject by administering a single-domain antibody (sdAb) comprising the amino acid sequence set forth in SEQ ID NO:1.

In certain embodiments, the sdAb is administered systemically at a therapeutically effective dose, including about 10 mg/kg body weight, and inhibits STAT3 activation in CD4+ T-cells, thereby reducing Th17-mediated inflammation. The methods are applicable to subjects in need thereof, including human subjects, and may be used to treat autoimmune forms of uveitis, such as sympathetic ophthalmia, birdshot retinochoroidopathy, Behcet's disease, Vogt-Koyanagi-Harada disease, and ocular sarcoidosis.

In some embodiments, the sdAb is administered alone or in combination with one or more anti-inflammatory or immunosuppressive agents. The sdAb may be administered according to various dosing regimens, including once-daily or twice-daily administration.

In certain aspects, administration of the sdAb preserves or improves retinal function, as measured by electroretinogram (ERG) amplitude relative to untreated control or baseline.

The disclosure also provides methods of modulating immune responses associated with uveitis by administering the sdAb, wherein the sdAb inhibits STAT3 phosphorylation in CD4+ T-cells and reduces production of IL-17. In some embodiments, the methods reduce expansion of Th17 cells and decrease levels of cytokines associated with inflammatory responses, including IL-17, IFN-7, GM-CSF, and IL-23. In further embodiments, the sdAb penetrates lymphocytes and inhibits intracellular STAT3 signaling, thereby reducing the uveitogenic potential of T-cells.

In additional aspects, the disclosure provides pharmaceutical compositions comprising the sdAb and a pharmaceutically acceptable carrier. Suitable carriers include, but are not limited to, phosphate-buffered saline (PBS), saline, water, and formulations comprising serum albumin. The compositions may be formulated as solutions or suspensions and may be configured for systemic or ocular administration.

These and other aspects of the invention provide improved methods for treating uveitis and related inflammatory conditions by targeting intracellular STAT3 signaling pathways and modulating pathogenic immune cell populations.

### DRAWINGS

These and other features, aspects, and advantages of the present invention will become better understood with regard to the following description, appended claims, and accompanying drawings where:

FIG. 1A depicts the results of a T-cell proliferation assay using a [³H]-thymidine incorporation assay, FIG. 1B depicts the results of Human Jurkat T-cells proliferation assay using a [³H]-thymidine incorporation assay;

FIG. 2 depicts graphical representations of C57BL/6J mice immunized with IRBP in CFA and treated with PBS or SBT-100 (SEQ ID NO:1) and development of EAU was assessed by fundoscopy (2A), histology (2B), or ERG (2C);

FIG. 3 shows graphical representations of C57BL/6J mice with induced EAU with (A) intracellular cytokine staining of Th1 or Th17 cells in the retina, DLN or spleen, (B) percentage of CD4⁺ T-cells expressing ROR-γT (B) or Granzyme B (C) in the retina, DLN or spleen, (D) ELISA results of sorted CD4⁺ T-cells, and (E) intracellular cytokine staining cells from the spleen of the EAU mice;

FIG. 4 depicts graphical representations of spleen cells from PBS-treated or SBT-treated mice with EAU and disease was assessed by fundoscopy (A), histopathology (B), and ERG (C); and

FIG. 5 depicts CD4+ T-cells that were analyzed by (A) by intracellular cytokine staining assay, (B) ELISA, (C) intracellular cytokine staining assay, and (D) multiplex ELISA.

### DESCRIPTION

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

The term “antigenic determinant” refers to the epitope on the antigen recognized by the antigen-binding molecule (such as an sdAb or polypeptide of the invention) and more in particular by the antigen-binding site of the antigen-binding molecule. The terms “antigenic determinant” and “epitope” may also be used interchangeably. An amino acid sequence that can bind to, that has affinity for and/or that has specificity for a specific antigenic determinant, epitope, antigen or protein is said to be “against” or “directed against” the antigenic determinant, epitope, antigen or protein.

As used herein, the term “comprise” and variations of the term, such as “comprising” and “comprises,” are not intended to exclude other additives, components, integers or steps.

It is contemplated that the sdAbs, polypeptides and proteins described herein can contain so-called “conservative” amino acid substitutions, which can generally be described as amino acid substitutions in which an amino acid residue is replaced with another amino acid residue of similar chemical structure, and which has little or essentially no influence on the function, activity or other biological properties of the polypeptide. Conservative amino acid substitutions are well known in the art. Conservative substitutions are substitutions in which one amino acid within the following groups (a)-(e) is substituted by another amino acid within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp. Other conservative substitutions include: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

A “domain” as used herein generally refers to a globular region of an antibody chain, and in particular to a globular region of a heavy chain antibody, or to a polypeptide that essentially consists of such a globular region.

The amino acid sequence and structure of an sdAb is typically made up of four framework regions or “FRs,” which are referred to as “Framework region 1” or “FR1”; as “Framework region 2” or “FR2”; as “Framework region 3” or “FR3”; and as “Framework region 4” or “FR4,” respectively. The framework regions are interrupted by three complementarity determining regions or “CDRs,” which are referred as “Complementarity Determining Region 1” or “CDR1”; as “Complementarity Determining Region 2” or “CDR2”; and as “Complementarity Determining Region 3” or “CDR3,” respectively.

As used herein, the term “humanized sdAb” means an sdAb that has had one or more amino acid residues in the amino acid sequence of the naturally occurring VHH sequence replaced by one or more of the amino acid residues that occur at the corresponding position in a VH domain from a conventional 4-chain antibody from a human. This can be performed by methods that are well known in the art. For example, the FRs of the sdAbs can be replaced by human variable FRs.

As used herein, an “isolated” nucleic acid or amino acid has been separated from at least one other component with which it is usually associated, such as its source or medium, another nucleic acid, another protein/polypeptide, another biological component or macromolecule or contaminant, impurity or minor component.

The term “mammal” is defined as an individual belonging to the class Mammalia and includes, without limitation, humans, domestic and farm animals, and zoo, sports, and pet animals, such as cows, horses, sheep, dogs and cats.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, Ringer's solutions, dextrose solution, PBS (phosphate-buffered saline), and 5% human serum albumin. Liposomes, cationic lipids and non-aqueous vehicles such as fixed oils may also be used. The use of such media and agents for pharmaceutically active substances is well known in the art. Except insofar as any conventional media or agent is incompatible with a therapeutic agent as defined above, use thereof in the composition of the present invention is contemplated.

A “quantitative immunoassay” refers to any means of measuring an amount of antigen present in a sample by using an antibody. Methods for performing quantitative immunoassays include, but are not limited to, enzyme-linked immunosorbent assay (ELISA), specific analyte labeling and recapture assay (SALRA), liquid chromatography, mass spectrometry, fluorescence-activated cell sorting, and the like.

The term “solution” refers to a composition comprising a solvent and a solute, and includes true solutions and suspensions. Examples of solutions include a solid, liquid or gas dissolved in a liquid and particulates or micelles suspended in a liquid.

The term “specificity” refers to the number of different types of antigens or antigenic determinants to which a particular antigen-binding molecule or antigen-binding protein molecule can bind. The specificity of an antigen-binding protein can be determined based on affinity and/or avidity. The affinity, represented by the equilibrium constant for the dissociation of an antigen with an antigen-binding protein (KD), is a measure for the binding strength between an antigenic determinant and an antigen-binding site on the antigen-binding protein: the lesser the value of the KD, the stronger the binding strength between an antigenic determinant and the antigen-binding molecule (alternatively, the affinity can also be expressed as the affinity constant (KA), which is 1/KD). As will be clear to one of skill in the art, affinity can be determined depending on the specific antigen of interest. Avidity is the measure of the strength of binding between an antigen-binding molecule and the antigen. Avidity is related to both the affinity between an antigenic determinant and its antigen binding site on the antigen-binding molecule and the number of pertinent binding sites present on the antigen-binding molecule. Specific binding of an antigen-binding protein to an antigen or antigenic determinant can be determined by any known manner, such as, for example, Scatchard analysis and/or competitive binding assays, such as radioimmunoassays (RIA), enzyme immunoassays (EIA) and sandwich competition assays.

As used herein, the term “recombinant” refers to the use of genetic engineering methods (for example, cloning, and amplification) used to produce the sdAbs of the invention.

A “single domain antibody,” “sdAb” or “VHH” can be generally defined as a polypeptide or protein comprising an amino acid sequence that is comprised of four framework regions interrupted by three complementarity determining regions. This is represented as FR1-CDR1-FR2-CDR2-FR3-CDR3-FR4. An sdAb of the invention also includes a polypeptide or protein that comprises the sdAb amino acid sequence. Typically, sdAbs are produced in camelids such as llamas, but can also be synthetically generated using techniques that are well known in the art. As used herein, the variable domains present in naturally occurring heavy chain antibodies will also be referred to as “VHH domains,” in order to distinguish them from the heavy chain variable domains that are present in conventional 4-chain antibodies, referred to as “VH domains,” and from the light chain variable domains that are present in conventional 4-chain antibodies, referred to as “VL domains.” “VHH” and “sdAb” are used interchangeably herein. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113.

The term “synthetic” refers to production by in vitro chemical or enzymatic synthesis.

The term “target” as used herein refers to any component, antigen, or moiety that is recognized by the sdAb. The term “intracellular target” refers to any component, antigen, or moiety present inside a cell. A “transmembrane target” is a component, antigen, or moiety that is located within the cell membrane. An “extracellular target” refers to a component, antigen, or moiety that is located outside of the cell.

A “therapeutic composition” as used herein means a substance that is intended to have a therapeutic effect such as pharmaceutical compositions, genetic materials, biologics, and other substances. Genetic materials include substances intended to have a direct or indirect genetic therapeutic effect such as genetic vectors, genetic regulator elements, genetic structural elements, DNA, RNA and the like. Biologics include substances that are living matter or derived from living matter intended to have a therapeutic effect.

As used herein, the phrases “therapeutically effective amount” and “prophylactically effective amount” refer to an amount that provides a therapeutic benefit in the treatment, prevention, or management of a disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The specific amount that is therapeutically effective can be readily determined by an ordinary medical practitioner, and may vary depending on factors known in the art, such as, e.g., the type of disease, the patient's history and age, the stage of disease, and the administration of other therapeutic agents.

The present invention relates to the use of single-domain antibodies (sdAbs) that are directed against intracellular components, as well as to the use of proteins and polypeptides comprising the sdAbs and nucleotides encoding the proteins and polypeptides to treat or prevent disease. The invention also includes nucleic acids encoding the sdAbs, proteins and polypeptides, and compositions comprising the sdAbs.

The amino acid sequence of an anti-STAT3 sdAb, named SBT-100 (SEQ ID NO:1) (or VHH13) (SEQ ID NO:1), is shown below:

HVQLVESGGGSVQAGGSLRLSCAASGANGGRSCMGWFRQVPGKEREGVSG
ISTGGLITYYADSVKGRFTISQDNTKNTLYLQMNSLKPEDTAMYYCATSR
FDCYRGSWFNRYMYNSWGQGTQVTVSS

The three CDRs are underlined.

The corresponding anti-STAT3 SBT-100 DNA sequence (SEQ ID NO:2) is:

5′-catgtgcagctggtggagtctgggggaggctcggtgcaggctggagg
gtctctgagactctcctgtgcagcctctggagccaacggtggtcggagct
gcatgggctggttccgccaggttccagggaaggagcgcgagggggtttct
ggtatttcaaccggtggtcttattacatactatgccgactccgtgaaggg
ccgattcaccatctcccaagacaacaccaagaacacgctgtatctgcaaa
tgaacagcctgaaacctgaggacactgccatgtactactgtgcgacgagt
cggtttgactgctatagaggctcttggttcaaccgatatatgtataacag
ttggggccaggggacccaggtcactgtctcctca-3′

SBT-100 (SEQ ID NO:1) (SEQ ID NO:1) has been previously described in U.S. patent Ser. No. 14/922,093, the contents of which are incorporated herein by reference.

STAT3 activates transcription of genes that regulate cell growth, differentiation, and survival. Genetic deletion of Stat3 in T-cells abrogates Th17 differentiation, suggesting that STAT3 is a potential therapeutic target for Th17-mediated diseases. However, a major impediment to therapeutic targeting of intracellular proteins such as STAT3 is the lack of efficient methods for delivering STAT3 inhibitors into cells. In this study, a single domain antibody (sdAb), or nanobody (SBT-100) comprised of the variable (V) region of a STAT3-specific heavy chain molecule was used. It was found that SBT-100 (SEQ ID NO:1) (SEQ ID NO:1) enters human and mouse cells, induced suppression of STAT3 activation and lymphocyte proliferation in concentration dependent manner. To investigate whether SBT-100 (SEQ ID NO:1) (SEQ ID NO:1) would be effective in suppressing inflammation in vivo, experimental autoimmune uveitis was induced in C57BL/6J mice by active immunization with the ocular autoantigen, interphotoreceptor retinoid binding protein (IRBP). Analysis of the retina by fundoscopy, histological examination or optical coherence tomography showed that treatment of the mice with SBT-100 (SEQ ID NO:1) (SEQ ID NO:1) suppressed uveitis by inhibiting expansion of pathogenic Th17 cells that mediate EAU. Electroretinographic (ERG) recordings of dark and light adapted a- and b-waves showed that SBT-100 (SEQ ID NO:1) treatment rescued the mice from developing significant visual impairment that characterize EAU in untreated mice. Adoptive transfer of activated IRBP-specific T-cells from untreated EAU mice induced EAU, while EAU was significantly attenuated in mice that received IRBP-specific T-cells from SBT-100 (SEQ ID NO:1) treated mice. Taken together, these results demonstrate efficacy of SBT-100 (SEQ ID NO:1) in mice and suggests its therapeutic potential for human autoimmune diseases.

SBT-100 (SEQ ID NO:1) is a nanobody, or single domain antibody (sdAb), consisting of a single VHH derived from a camelid immunoglobulin heavy chain variable region devoid of light chain. SBT-100 (SEQ ID NO:1) can penetrate lymphocytes and inhibits IL-6/STAT3 signaling pathways of primary mouse CD4+ lymphocytes and human Jurkat T-cells. SBT-100 (SEQ ID NO:1) was also shown to be effective in vivo and suppresses the development of EAU by inhibiting the expansion of pathogenic Th17 cells. The use of single-domain antibodies (sdAbs) as single antigen-binding proteins or as an antigen-binding domain in larger protein or polypeptide offers a number of significant advantages over the use of conventional antibodies or antibody fragments. The advantages of sdAbs include: only a single domain is required to bind an antigen with high affinity and with high selectivity; sdAbs can be expressed from a single gene and require no post-translational modification; sdAbs are highly stable to heat, pH, proteases and other denaturing agents or conditions; sdAbs are inexpensive to prepare; and sdAbs can access targets and epitopes not accessible to conventional antibodies.

Since the sdAbs of the invention are mainly intended for therapeutic use, they are directed against mammalian, preferably human, targets. However, it is possible that the sdAbs described herein are cross-reactive with targets from other species, for example with targets from one or more other species of primates or other animals (for example, mouse, rat, rabbit, pig or dog), and in particular in animal models for diseases and disorders associated with the disease associated with the targets.

The invention further relates to applications and uses of the sdAb, the nucleic acids encoding the sdAbs, host cells, products and compositions described herein. Such a product or composition may, for example, be a pharmaceutical composition for treatment or prevention of a disease.

The present invention generally relates to sdAbs, as well as to proteins or polypeptides comprising or essentially consisting of one or more of such sdAbs, that can be used for prophylactic and therapeutic purposes.

The methods and compositions detailed in the present invention can be used to treat disease described herein, and can be used with any dosage and/or formulation described herein or otherwise known, as well as with any route of administration described herein or otherwise known to one of skill in the art.

The sdAb of the invention can be used with one or more compounds. The one or more compounds can increase the therapeutic response and augment the effectiveness of the sdAb of the invention. In addition, the effectiveness of the sdAb can be increased by combining it with peptides, peptidomimetics, and other drugs.

STAT3 is a member of the signal transducers and activators of transcription (STAT) family of proteins that carry both signal transduction and activation of transcription functions. STAT3 is widely expressed and becomes activated through phosphorylation on tyrosine and/or serine as a DNA binding protein in response to a various cytokines and growth factors such as EGF, IL-6, PDGF, IL-2 and G-CSF. The STAT3 phosphoprotein forms homodimers and heterodimers with other members of the STAT family and translocates to the nucleus in order to modulate the transcription of various genes, and as a result plays a key role in many cellular processes such as cell growth, apoptosis, angiogenesis, immune evasion, and survival.

Uveitis is a diverse group of potentially sight-threatening intraocular inflammatory diseases that is characterized by repeated cycles of remission and recurrent intraocular inflammation, and visual handicap is of significant public health importance as it affects patient's quality of life. Increased recruitment of Th17 cells into the retina is implicated in pathophysiology of uveitis and current therapies include periocular or intravitreal corticosteroid. However, their prolonged use for treatment of chronic uveitis is associated with development of serious side effects such as glaucoma and is the impetus for developing alternative therapies. Targeting the STAT3 pathway required for the differentiation and expansion of Th17 cells has been proposed as a potential therapy for mitigating uveitis because genetically modified mice that cannot induce Th17 cells are resistant to developing uveitis. However, a major impediment to targeting STAT3 pathway is that it is an intracellular protein and not accessible to STAT3-specific antibodies, as well as the unpredictable pharmacokinetic characteristics of small molecular weight STAT3 inhibitory peptides or mimetics.

In contrast to the conventional STAT3-specific antibodies composed of heterotetrameric immunoglobulins assembled from two identical heavy (H)-chain and two identical light (L)-chain polypeptides, the STAT3-specific nanobody used to target STAT3 signaling pathway in this study is a unique camelid-like single-domain monomeric VHH antibody comprised of a unique one antigen-binding domain. In contrast to 90 kDa nanobodies that do not penetrate cells, the STAT3 VHH is ~15 kDa (2.5 nm) in size which allows it to penetrate cells and is not toxic to tissues. Uveitis was induced in mice, the mice were then treated twice daily with SBT-100 (SEQ ID NO:1) and analysis of the eyes by fundoscopy, histology, optical coherence tomography and electroretinography revealed that SBT-100 (SEQ ID NO:1) confers protection from severe uveitis and suppressed ocular inflammation by inhibiting Th1 and Th17 responses, curtailed expansion and trafficking of inflammatory cells into retina during EAU. The results show that SBT-100 (SEQ ID NO:1) is not toxic to ocular cells, and it prevented decrement of retina function usually associated with severe ocular inflammation. Consistent with the well establish role of T-cells in etiology of uveitis, the results show that transfer of cells from EAU mice that were treated with SBT-100 (SEQ ID NO:1) induced very mild EAU compared to adoptive transfer of cells from untreated EAU mice that developed full-blown disease. Taken together these results provide suggestive evidence that SBT-100 (SEQ ID NO:1) immunotherapy is effective in ameliorating uveitis in humans.

Central nervous system (CNS) autoimmune diseases such as uveitis and multiple sclerosis result as consequence of breakdown of immune privilege of the brain, spinal cord or neuroretina which are maintained by the blood-retina barrier (BRB), blood-brain-barrier (BBB) and the neurovascular unit (NVU) comprised of pericytes, perivascular macrophages, tightly bound endothelial cells, glia limitans of the Müller/microglia. These structures sequester CNS tissues from peripheral immune system and Th17 cells that produce Granzyme B are implicated in early events that initiate CNS autoimmune diseases by promoting the disruption of the BBB or BRB. However, sustained activation of microglial cells and recruitment of other inflammatory cells amplify the inflammatory response and are responsible for pathology characteristic of chronic uveitis or multiple sclerosis. Nonetheless, interventional studies using biologics such as cytokines or immune-suppressive compounds to suppress uveitis in mice invariably show strong correlation of disease amelioration with suppression of pathogenic Th17 cells. Subsequent studies revealed the requirement of STAT3 for Th17 differentiation and development while others showed that targeted deletion of STAT3 prevented the development of EAE or EAU. These studies led to the now established notion that targeting Th17 cells is a viable therapeutic approach for suppressing and mitigating autoimmune and autoinflammatory diseases.

In this study, SBT-100 (SEQ ID NO:1) immunotherapy conferred protection against EAU by antagonizing the expansion of the pathogenic Th1 and Th17 cells as well as Treg cells. While Th17 cells play important role in initiating the disease, the data suggests that therapeutics designed to inhibit Th17 would only be partially effective. The data is consistent with previous studies showing that STAT3 has a dual role in T-cells: it plays the important role of maintaining unactivated T-cells at the G₀ phase as resting cells by inhibiting IL-2 production through up-regulation of the lymphocyte quiescence Class O forkhead transcription factors. It is of note that, while STAT3 maintains a T-cell as resting cells, after engaging cognate antigen and entry into the G₁ cell cycle phase, STAT3 promotes cell proliferation as is the case in all mammalian cells. Thus, the STAT3-specific nanobody mediates suppression of Th17, Th1, Treg lymphocytes as well as inflammatory myeloid cells that perpetuate neuroinflammation.

Uveitis is a group of syndromic diseases which includes sympathetic ophthalmia, birdshot retinochoroidopathy, Behcet's disease, Vogt-Koyanagi-Harada disease and ocular sarcoidosis. It accounts for more than 10% of severe visual handicaps in the United States and major impediment to treatment of the disease with antibodies is their size that restricts entry into the CNS because of the BRB and the neuroretinal vascular unit. The results show that SBT-100 (SEQ ID NO:1) can cross the BRB, treat an organ specific autoimmune ophthalmic disease, and significantly inhibit both TH17 and TH1 cells in vivo. Moreover, SBT-100 (SEQ ID NO:1) is non-toxic and readily enters CNS tissues such as the brain, spinal cord and the neuroretina.

### EXAMPLES

### Example 1: SBT-100 (SEQ ID NO:1) Suppresses T-Cell Proliferation and STAT3 Activation in Primary T-Cells

Six- to eight-week old C57BL/6J mice were purchased from Jackson Laboratory (Jackson Laboratory, Bar Harbor, ME). Animals were housed at the NIH/NEI animal facility, maintained under light-dark cycle with unlimited access to water and chow. All animal care and procedures were humane and conformed with the National Institute of Health Animal Care and Use Committee guidelines.

Jurkat-cells, Clone E6-1 (ATCC® TIB-152™) were obtained from ATCC (Gaithersburg, MD). All cells were cultured in complete RPMI 1640 media (supplemented with fetal bovine serum (FBS) to a final concentration of 10% and 1×Penicillin-Streptomycin, 2 mM L-glutamine (Life Technologies, Grand Island, NY), 5 μM 2-mercaptoethanol) in a humidified incubator at 37° C.

Plasma cytokines were quantified by multiplex ELISA. Plasma was separated by centrifugation at 1,000×g for 10 mins and cytokines quantified using LEGENDplex Mouse Inflammation panel as recommended by manufacturer (BioLegend, San Diego, CA). Data acquisition was performed on CytoFLEX Flow Cytometer (Beckman Coulter, Indianapolis, IN) and analyzed using BioLegend's LEGENDplex™ data analysis software.

Previous studies have shown that activation of STAT3 pathway regulates T-cell proliferation and Th17 differentiation while loss of STAT3 in T-cells prevents development of CNS autoimmune diseases. In this study, an antibody was developed to penetrate membranes, blood-brain barrier (BBB) and the blood retina barrier (BRB) in order to examine whether this camelid-derived nanobody, SBT-100, would be effective in suppressing Th17-induced autoimmune diseases. SBT-100 (SEQ ID NO:1) is a single domain, 2.5 nm, 15 kDa antibody comprised of a single monomeric variable antibody domain and lacks the light chain and CH domain of the heavy chain normally present in conventional Fab region. To determine whether SBT-100 (SEQ ID NO:1) can enter the cytoplasm of primary T-cells and antagonize STAT3 signaling, cells were isolated from the spleen of C57BL/6J mice, and sorted T-cells using CD4-specific magnetic beads. The cells were shown to be CD4⁺ lymphocytes by FACS analysis.

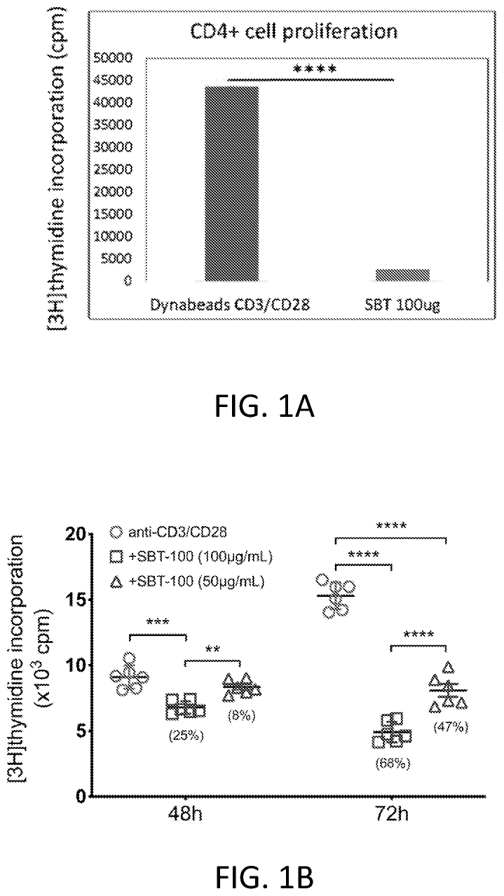

To determine whether SBT-100 (SEQ ID NO:1) can inhibit STAT3 activation, the cells were stimulated with anti-CD3/CD28 for 3 days in medium containing SBT-100 (SEQ ID NO:1) (100 μg/ml or 50 μg/ml). As seen in FIG. 1, SBT-100 (SEQ ID NO:1) inhibits lymphocyte and STAT3 activation. Analysis of the cells by [³H]-thymidine incorporation and lymphocyte proliferation assay revealed significant inhibition of T-cell proliferation (FIG. 1A). Sorted mouse primary naïve CD4+ T-cells were stimulated with anti-CD3/CD28 in medium containing SBT-100 (SEQ ID NO:1) and on day 3 T-cell proliferation was assessed by [3H]-thymidine incorporation assay.

For evaluating STAT3 activation, after 48 hours of stimulation with anti-CD3/anti-CD28, the cells were washed, starved for 2 hours in serum-free medium containing 0.5% BSA and re-stimulated for 30 min with IL-6. pSTAT3 was detected by Western blot. Cell extracts (20-40 μg/lane) were fractionated on 4-12% gradient SDS-PAGE in reduced condition and Western blot analysis was performed using antibodies specific to mouse STAT3, pSTAT3 or b-Actin (Cell Signaling Technology, Danvers, MA). The primary antibodies were detected using anti-mouse-IRDye 680RD and anti-rabbit-IRDye 800RD secondary antibodies for the Li-Cor two color system which allows detection of two targets on the same membrane. Each Western blotting analysis was repeated at least three times.

Western blot analysis of the lysates revealed that in the absence of SBT-100, the IL-6 activated STAT3 (pSTAT3), while addition of SBT-100 (SEQ ID NO:1) suppressed pSTAT3 activation in a concentration dependent manner (data not shown). Additionally, the inhibitory effect of SBT-100 (SEQ ID NO:1) suppressed proliferation of human Jurkat T-cells as indicated by [³H]-thymidine incorporation and lymphocyte proliferation assay (FIG. 1B). Human Jurkat T-cells were stimulated for 72 h with anti-CD3/CD28 in medium containing SBT-100 (SEQ ID NO:1) and on day 3 T-cell proliferation was assessed by [3H]-thymidine incorporation assay. Data represent at least 3 independent experiments and presented as mean±SEM. (**p<0.01; **p<0.001; ****p<0.0001).

### Example 2: SBT-100 (SEQ ID NO:1) Ameliorates Uveitis and Preserves Vision During Intraocular Inflammation

Experimental Autoimmune Uveitis (EAU) was induced in C57BL/6J mice by active immunization with IRBP651-670-peptide (300 μg per mouse) in a 200 μL emulsion (1:1 v/v) with complete Freund's adjuvant (CFA) containing Mycobacterium tuberculosis strain H37RA (2.5 mg/mL) subcutaneously as previously described (Oh, H. M., et. al., Journal of Biological Chemistry, 287, 30436-30443 (2012). Mice also received intraperitoneal injection of Bordetella pertussis toxin (1 μg/mouse) concurrently with immunization. Starting from Day −1 of immunization to Day 12 post immunization, mice were treated twice daily with either 100 μL PBS or SBT-100 (SEQ ID NO:1) (10 mg/kg body weight in 100 μL PBS). For each study, 8 mice were used per group and matched by age and sex.

To characterize infiltrating inflammatory cells in the retina of EAU mice, mice were euthanized and perfused with PBS as described (Oh et al, 2012). Enucleated eyes were put in petri dish containing culture medium and the retina isolated under a dissecting microscope by cutting along the limbus and lens, and cornea carefully removed. The retina was then peeled off and transferred to RPMI media containing collagenase (1 mg/mL) and DNase (10 μg/mL) for 2 hours at 37° C. The digesting tissue was periodically pipetted every 30 mins to enhance tissue digestion. The digestion was stopped by adding 10 folds volume of complete medium. The cells were then washed twice with complete medium and cells counted using the Vi-Cell XR cell viability analyzer (Beckman Coulter).

Clinical disease was established and scored by fundoscopy and histology as described previously (Oh et al, 2012). Funduscopic examinations were performed at day 10 to 21 after EAU induction. Following administration of systemic anesthesia (intraperitoneal injection of ketamine (1.4 mg/mouse) and xylazine (0.12 mg/mouse)), the pupil was dilated by topical administration of 1% tropicamide ophthalmic solution (Alcon Inc., Fort Worth, TX). Fundus image was captured using Micron III retinal imaging microscope (Phoenix Research Labs) for small rodent or a modified Karl Storz veterinary otoendoscope coupled with a Nikon D90 digital camera, as previously described (Oh, H. M. et al., Journal of Immunology, 187, 3338-3346 (2011)). At least 6 images (2 posterior central retinal view, 4 peripheral retinal views) were taken from each eye by positioning the endoscope and viewing from superior, inferior, lateral and medial fields and each individual lesion was identified, mapped and recorded. The clinical grading system for retinal inflammation was as established (Oh et al, 2012).

Eyes were examined for disease severity using binocular microscope with coaxial illumination. Eyes for histology were enucleated 20 days post-immunization, fixed in 10% buffered formalin, and serially sectioned in the vertical pupillary-optic nerve plane. All sections were stained with hematoxylin and eosin.

EAU is a predominantly T-cell-mediated CNS autoimmune disease and a well-characterized mouse model of human uveitis. This mouse model was used to investigate whether SBT-100 (SEQ ID NO:1) was effective in suppressing the development or severity of this organ-specific CNS autoimmune disease. EAU was induced in C57BL/6J mice by immunization with IRBP651-670 in CFA emulsion as previously described (Mattapallil, M. J. et al. Invest Ophthalmol Vis Sci 56, 5439-5449 (2015)). Mice were treated twice daily with either PBS (untreated group) or SBT-100 (SEQ ID NO:1) from Day −1 to Day 12 post EAU induction. In the EAU model, uveitis generally manifests between day 13 and day 22 post-immunization (p.i), therefore progression and severity of the disease was monitored during this period by fundoscopy, histology, optical coherence tomography (OCT) and electroretinography (ERG). EAU clinical scores and assessment of disease severity were based on changes at the optic nerve disc or retinal vessels and detection of retinal and choroidal infiltrates in the eye.

Statistical data analysis and graphs plots were performed using GraphPad Prism 8, using two-tailed unpaired Student's t test for pairwise comparisons or One-way ANOVA with multiple pairwise t test, depending on the experiments. Data are shown as mean and SD and statistical significance for inferences was based on p<0.05. Data represent at least 3 independent experiments and presented as mean±SEM. (*p<0.05; **p<0.01; ****p<0.0001).

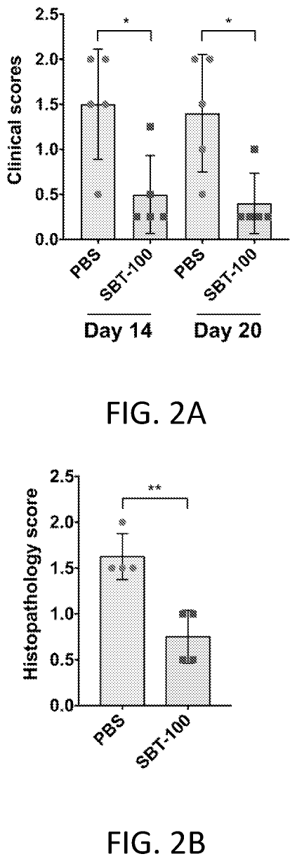

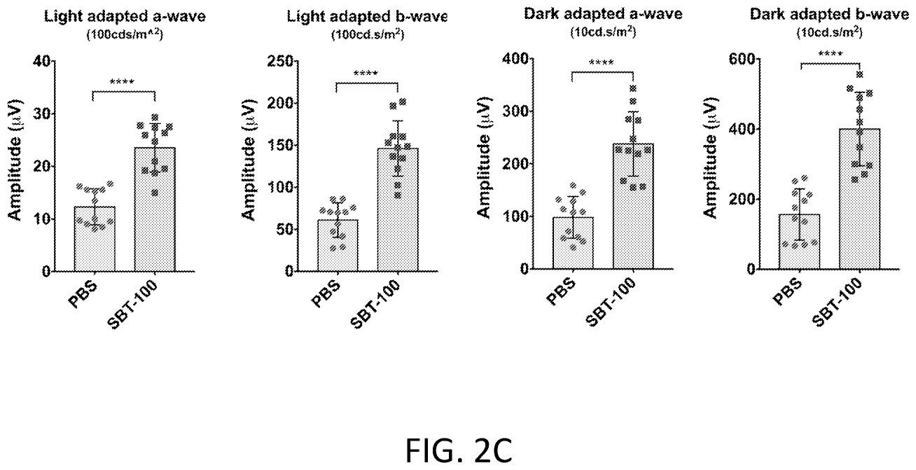

C57BL/6J mice were immunized with IRBP in CFA and treated with PBS or SBT-100 (SEQ ID NO:1) and development of EAU was assessed by fundoscopy (FIG. 2A), histology (FIG. 2B), OCT (data not shown) or ERG (FIG. 2C).

The initial signs of uveitis were first observed by day 14 p.i., and full-blown inflammation was observed between then and day 20 p.i. Fundus images of the PBS-treated retinae show severe inflammation characterized by blurred optic disc margins and enlarged juxtapapillary area, retinal vasculitis with moderate cuffing, and yellow-whitish retinal and choroidal infiltrates (data not shown). In contrast, fundus images of the SBT-100-treated mice retinae indicated a relatively mild EAU with very low clinical scores (data not shown). Clinical scores and assessment of disease severity were based on changes at the optic nerve disc or retinal vessels and retinal and choroidal infiltrates. Histopathology of a cross section of the eye was performed on Day 20 post immunization. There were characteristic extensive retinal lesions with some confluent lesions due to inflammatory cell infiltration, blurry optic disc margin and vasculitis in the untreated EAU control mice, all of which were mild or absent in the SBT-100 (SEQ ID NO:1) treated mice. EAU score was significantly higher in the PBS-treated group as compared to the SBT-100 (SEQ ID NO:1) treated group. Consistent with fundoscopy results, histology of the PBS-treated day 20 retina revealed severe EAU with infiltration of large numbers of inflammatory cells into the vitreous, destruction of retinal cells and development of retinal in-folding, a hallmark of severe uveitis.

OCT is a noninvasive procedure that allows visualization of internal microstructure of various eye structures in living animals and was performed as previously described (Oh et al, 2012). A Spectral-domain optical coherence tomography (SD-OCT) system with 820 nm center wavelength broadband light source (Bioptigen, NC) was used for in vivo non-contact imaging of eyes from control or EAU mice. Mice were anesthetized and the pupils dilated. Mice were then immobilized using adjustable holder that could be rotated easily allowing for horizontal or vertical scanning and each scan was performed at least twice, with realignment each time. The dimension of the scan (in depth and transverse extent) was adjusted until the optimal signal intensity and contrast was achieved. Retinal thickness was measured from the central retinal area of all images obtained from both horizontal and vertical scans from the same eye, using the system software, and averaged. The method used to determine the retinal thicknesses in the system software was as described (Gabriele, M. L. et al., Invest Ophthalmol Vis Sci 52, 2250-2254 (2011)).

OCT revealed accumulation of inflammatory cells in the vitreous and optic nerve head of the PBS-treated mice but not the retina of mice that received SBT-100 (SEQ ID NO:1) (FIG. 2B). OCT images show accumulation of infiltrating cells around the optic nerve and damage to the optic disc (data not shown). Light-adapted (100 cd·s/m²) and dark adapted (10 cd·s/m²) a- and b-waves on Day 18 post immunization that were significantly lower in the untreated mice compared to the SBT-100 (SEQ ID NO:1) treated group.

Electroretinogram (ERG) is a well-established clinical method for detecting alterations in visual function during intraocular inflammation and is based on changes in electrical potential in response to light stimulation of the retina. ERG under light-adaptive stimuli reflect cone-driven functions while dark-adapted b-wave responses represent rod-driven activities. Before the ERG recordings, mice were dark-adapted overnight, and experiments were performed under dim red illumination as previously described (Oh et al, 2012). Mice were anesthetized with a single intraperitoneal injection of ketamine (1.4 mg/mouse) and xylazine (0.12 mg/mouse) and pupils were dilated with Mydriacyl containing of 0.5% tropicamide and 0.5% phenylephrine hydrochloride (Santen Pharmaceutical Co., Osaka, Japan). ERGs were recorded using an electroretinography console (Espion E2; Diagnosys LLC, Lowell, MA) that generated and controlled the light stimulus. Dark-adapted ERG was recorded with single flash delivered in a Ganzfeld dome with intensity of −4 to 1 log cd·s/m² delivered in 7 steps. Light-adapted ERG was obtained with a 10 cd·s/m² background, and light stimuli started at 0.3 to 100 cd·s/m² in 6 steps. Gonioscopic prism solution (Alcon Labs, Fort Worth, TX) was used to provide good electrical contact and to maintain corneal moisture. A reference electrode (gold wire) was placed in the mouth, and a ground electrode (subcutaneous stainless-steel needle) was positioned at the base of the tail. Signals were differentially amplified and digitized at a rate of 1 kHz. Amplitudes of the major ERG components (a- and b-wave) were measured (Espion software; Diagnosys LLC) using automated and manual methods. Immediately after ERG recording, imaging of the fundus was performed as previously described (Oh, H. M. et al, J. Immunology, 2011).

The significantly lower a- and b-wave amplitudes detected in eyes of PBS-treated mice under light- or dark-adapted condition, suggest significant visual impairment in PBS-treated mice while higher a- and b-wave amplitudes detected in SBT-treated mice are consistent with SBT-100 (SEQ ID NO:1) mediated preservation of vision during uveitis (FIG. 2C).

### Example 3: SBT-100 (SEQ ID NO:1) Suppressed Uveitis in Mice by Inhibiting Pathogenic Th1 and Th17 Cells

Uveitis in mice and humans is thought to be mediated primarily by T-cells of the Th17 subset and although the Th1 subset is also increased in the eyes of mice with EAU, its role in the etiology of uveitis is still a matter of debate. To investigate whether the amelioration of EAU observed in this study derived from SBT-100-induced decrease in levels of Th17 and/or Th1 cells, cells were isolated from mice with EAU. The frequency of Th17 and Th1 cells were examined in the eyes, draining lymph nodes and spleens of SBT-100 (SEQ ID NO:1) treated mice.

EAU was induced in C57BL/6J mice that were immunized with IRBP in CFA and treated with PBS or SBT100. Single cell suspensions of draining lymph nodes (DLN) and spleen DLNs were made. Spleens were dissected, and cells freed by teasing in a 40 μm pore cell strainer. Following washing in RPMI 1640 medium, erythrocytes were lysed using 5 mL of ACK RBC lysis buffer (Quality Biological, MD) for 3 mins. The lysis was stopped by adding 10× volume of the medium. Following 2 washes, cells were resuspended and seeded at a concentration of 2×10⁶/mL.

For intracellular cytokine detection, cells were re-stimulated for 5 h with PMA (50 ng/ml)/ionomycin (500 ng/ml). GolgiPlug was added in the last three hour and intracellular cytokine staining was performed using BD Biosciences Cytofix/Cytoperm kit as recommended (BD Pharmingen, San Diego, CA). FACS analysis was performed on a MACSQuant analyzer (Miltenyi Biotec, San Diego, CA) using protein-specific monoclonal antibodies and corresponding isotype control Abs (BD Pharmingen, San Diego, CA) as previously described (Oh et al, 2012). FACS analysis was performed on samples stained with mAbs conjugated with fluorescent dyes and each experiment was color compensated. Dead cells were stained with dead cell exclusion dye (Fixable Viability Dye eFluor® 450; eBioscience). Gates were set using isotype controls.

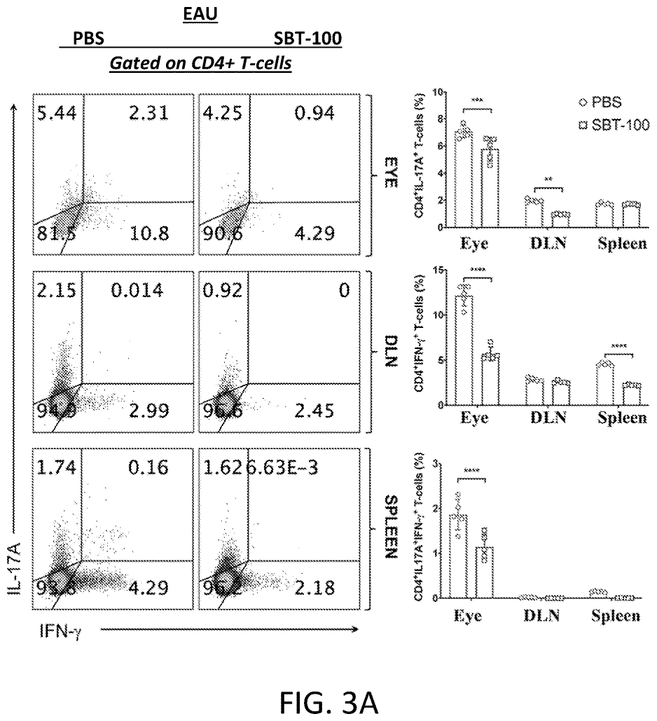

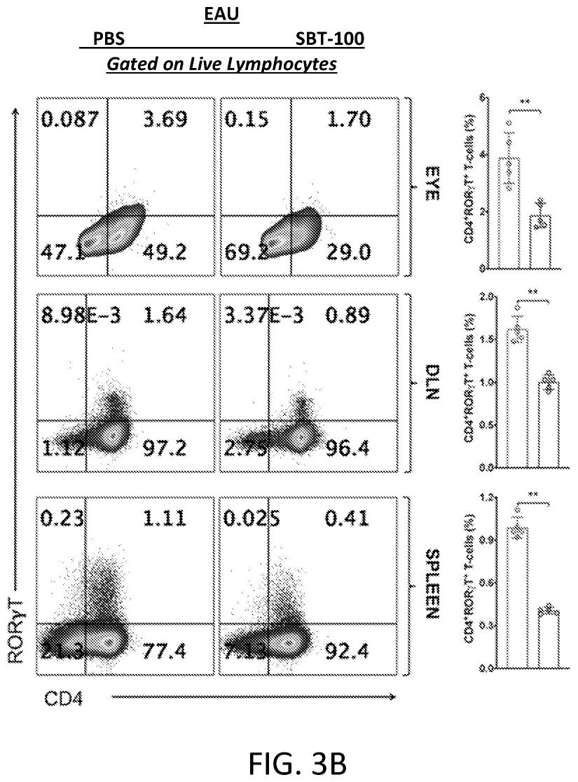

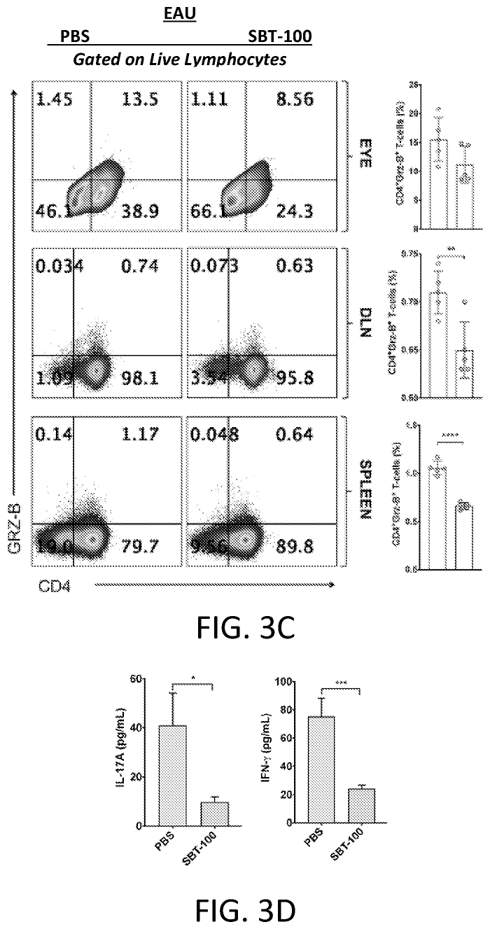

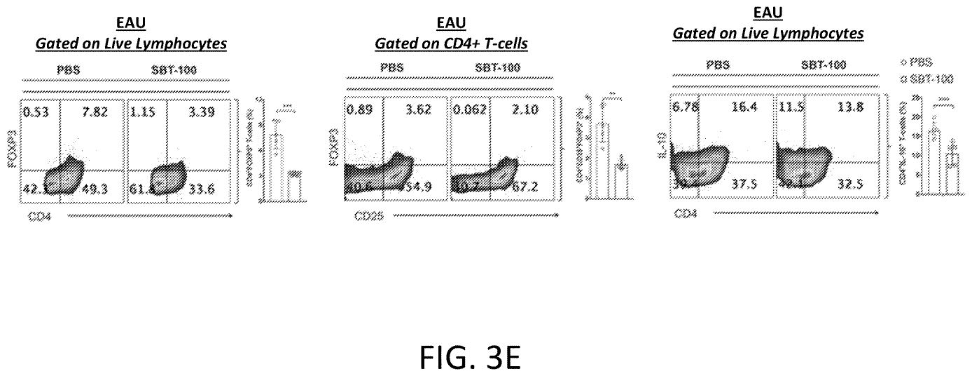

The frequency of CD4+ T-cells secreting proinflammatory IL-17 and/or IFN-7 cytokines were detected and quantified by the intracellular cytokine staining assay. Consistent with published reports, development of EAU in PBS-treated mice was accompanied by significant increase of IL-17-expressing (Th17) and IFNγ-expressing (Th1) cells in the eyes, spleen, and DLN while much lower percentages of Th1 and Th17 in these tissues of mice treated with SBT-100 (SEQ ID NO:1) was observed (FIG. 3A). Quadrants indicate percentage of CD4+ T-cells expressing IFN-7 or IL-17. The percentage of CD4+ T-cells expressing ROR-γT (FIG. 3B) or Granzyme B (FIG. 3C) in the retina, DLN or spleen (FIG. 3D). Sorted CD4+ T-cells from EAU mice treated with PBS or SBT-100 (SEQ ID NO:1) were re-stimulated in vitro with IRBP and IL-17A and IFN-γ secreted in day 3 culture supernatant was detected by ELISA.

These results are in line with studies implicating Th17 cells in pathogenesis of organ-specific autoimmune diseases. Th17 differentiation and development requires ROR-γt transcription, and the results show that the decrease in Th17 cells correlated with detection of lower levels of this Th17 master transcription factor (FIG. 3B). Th17 lymphocytes traffic to the central nervous system, mediate cytolytic effects via Granzyme B release and blocking Granzyme B ameliorates late/chronic EAE. Consistent with the curative effect of SBT-100, marked reduction of CD4 T-cells producing Granzyme B in mice treated with SBT-100 (SEQ ID NO:1) compared to the untreated mice was observed (FIG. 3C).

Measurement of IL-17 and IFN-γ in the supernatant of cultured CD4 T-cells from DLN was consistent with reduced levels of these cytokines in the SBT-100 (SEQ ID NO:1) treated mice (FIG. 3D). Cells from the spleen of the EAU mice treated with PBS or SBT-100 (SEQ ID NO:1) were analyzed by the intracellular cytokine staining (FIG. 3E). Quadrants indicate percentage of CD4+ T-cells expressing Foxp3, CD25 and/or IL-10. Data represent at least 3 independent experiments and presented as mean±SEM. (*p<0.05; **p<0.01; ***p<0.001; ****p<0.0001). Surprisingly, it was found that SBT-100-mediated inhibition of EAU is not associated with increase of regulatory T-cells (FIG. 3E).

### Example 4: SBT-100 (SEQ ID NO:1) Suppresses Uveitis Induced by Adoptive Transfer of Uveitogenic Lymphocytes

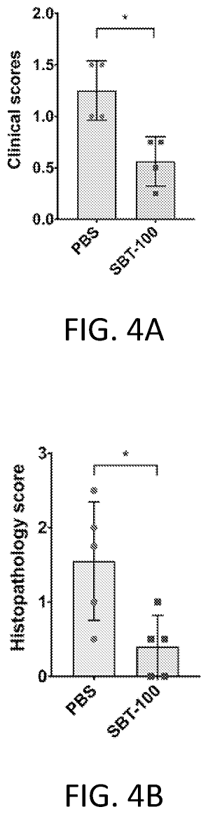

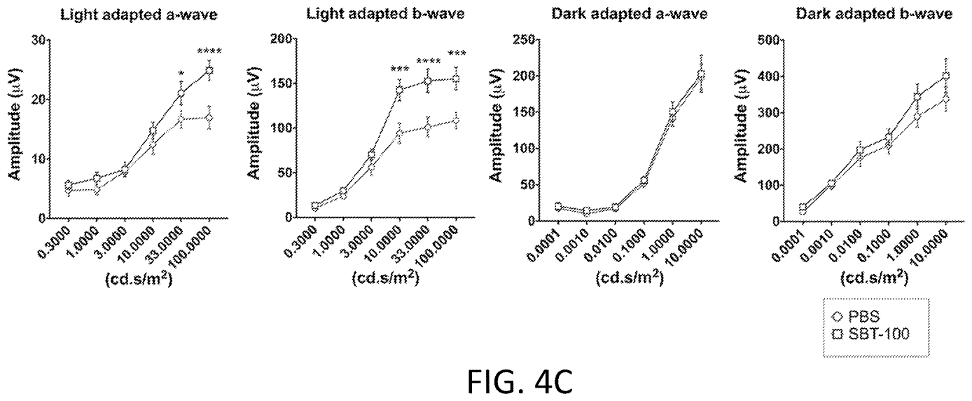

Adoptive transfer experiments were performed to directly investigate whether SBT-100 (SEQ ID NO:1) induced protection against EAU is a direct consequence of targeting STAT3 pathway in T-cells. Cells were isolated from lymph nodes and spleen of control or SBT-100-treated mice with EAU, re-stimulated the cells ex vivo with of IRBP₆₅₁₋₆₇₀ and 3×10⁷ cells were adoptively transferred to naïve WT C57BL/6J mice. Disease progression was assessed and EAU scores were determined 12 days after transfer of the cells by fundoscopy, histology, OCT, and ERG. Significant infiltration of inflammatory cells and vasculitis in mice that received cells from PBS-treated mice was observed, while mice that received cells from the SBT-100 (SEQ ID NO:1) treated mice were protected from severe EAU (FIG. 4A). Retinal infolding and cellular infiltration were also reduced in SBT-100 (SEQ ID NO:1) treated mice (FIG. 4B). OCT images also show significant accumulation of infiltrating cells around the optic nerve and distortion of retinal layers (data not shown). Significantly higher light-adapted a- and b-waves, as well as substantial increase of dark-adapted b-waves were detected in eyes of mice that received cells from SBT-100 (SEQ ID NO:1) treated mice compared to mice that received cells from untreated mice (FIG. 4C). Data represent at least 3 independent experiments and presented as mean±SEM. (*p<0.05; **p<0.01; ***p<0.001; ****p<0.0001). These results indicate that STAT3 pathway is a potential therapeutic target for ameliorating uveitis and preserved visual functions.

### Example 5: SBT-100 (SEQ ID NO:1) Antagonized Expansion of Pathogenic Th1 and Th17 Cells that Mediate Uveitis

Like the induction of EAU by active immunization with IRBP/CFA, transfer of T-cells treated with SBT-100 (SEQ ID NO:1) were inefficient in transferring EAU compared to control untreated cells. For detection of cytokine secretion by ELISA, cells were specifically re-stimulated for 48 h with IRBP₆₅₁₋₆₇₀ peptide and the supernatants collected were quantified using Quantikine ELISA as recommended by manufacturer.

Plasma cytokines were quantified by multiplex ELISA. Plasma was separated by centrifugation at 1,000×g for 10 mins and cytokines quantified using LEGENDplex Mouse Inflammation panel as recommended by manufacturer (BioLegend, San Diego, CA). Data acquisition was performed on CytoFLEX Flow Cytometer (Beckman Coulter, Indianapolis, IN) and analyzed using BioLegend's LEGENDplex™ data analysis software.

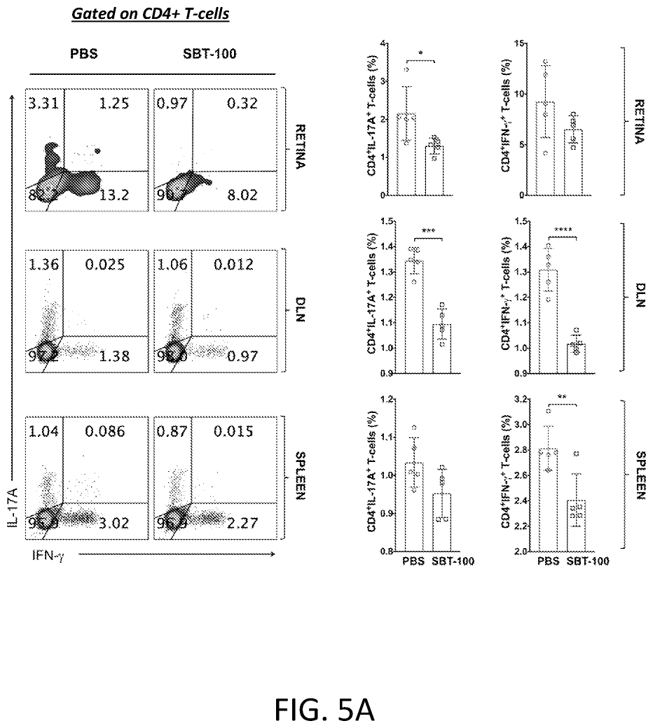

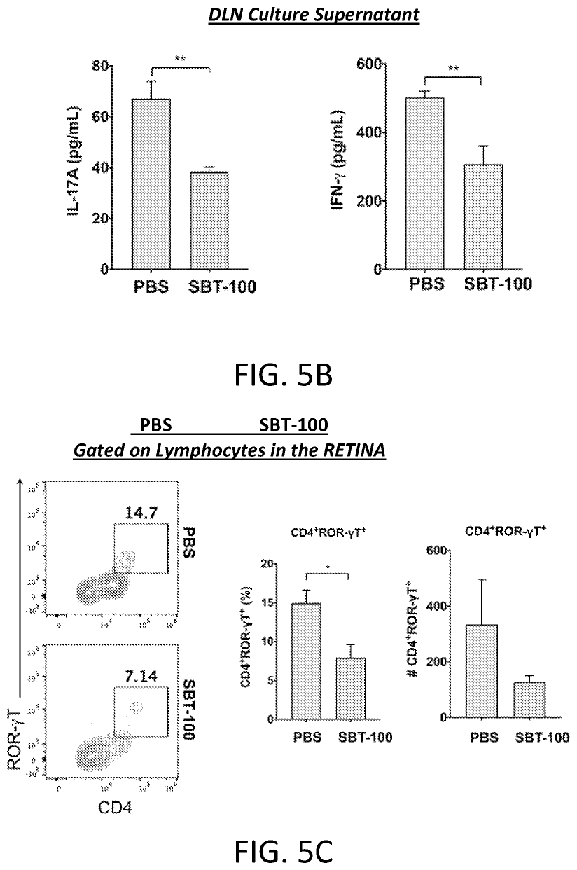

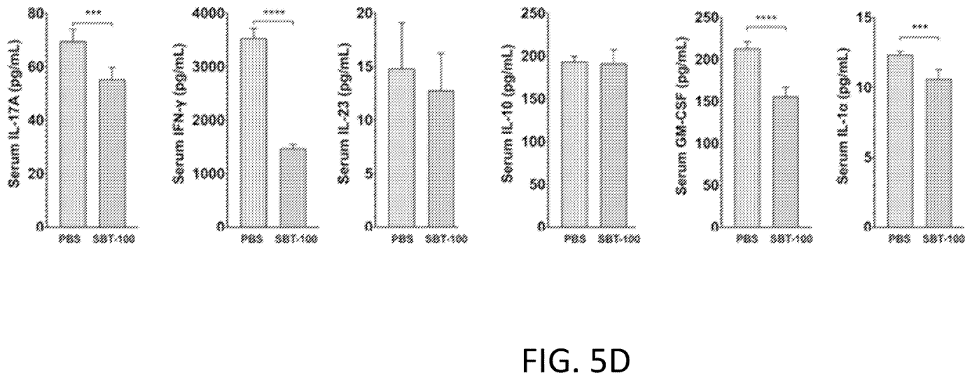

Quadrants indicate percentage of CD4+ T-cells expressing IFN-γ or IL-17 in the retina, DLN or spleen and Th1 or Th17 cells was determined by the intracellular cytokine staining. Intracellular cytokine staining analysis show significantly reduced levels of pathogenic Th1 and Th17 cells in mice that received cells from mice treated with SBT-100 (SEQ ID NO:1) (FIG. 5A). Sorted CD4+ T-cells from EAU mice treated with PBS or SBT-100 (SEQ ID NO:1) were re-stimulated in vitro with IRBP and IL-17A and IFN-γ secreted in day 3 culture supernatant was detected by ELISA. SBT-100 (SEQ ID NO:1) induced EAU amelioration correlated with decrease of Th1 and Th17 signature cytokines, IFN-γ and IL-17, respectively (FIG. 5B). Quadrants indicate percentage of CD4+ T-cells expressing ROR-gT in the retina. Consistent with the well-established role of Th17 cell in etiology of several autoimmune diseases, the percentage of T-cells expressing the Th17 master transcription factor, (ROR-γt) was significantly reduced (FIG. 5C). Plasma levels of IL17A, IFN-γ, GM-CSF, IL-1α and IL-10 were all detected in PBS-treated and SBT-100-treated mice by multiplex ELISA. Analysis of the serum of mice that received cells from untreated or SBT-100-treated mice revealed that the serum from SBT-100 (SEQ ID NO:1) treated group has significant reduction of proinflammatory cytokines including IL-17, IFN-γ, IL23, GM-CSF, and IL-1a, providing further suggestive evidence that SBT-100 (SEQ ID NO:1) treatment suppressed EAU (FIG. 5D). Data represent at least 2 independent experiments and presented as mean±SEM. (*p<0.05; **p<0.01; ***p<0.001; ****p<0.0001).

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claim should not be limited to the description of preferred embodiments contained in this disclosure. All references cited herein are incorporated by reference in their entirety.

What is claimed is:

1. A method of treating uveitis in a subject, comprising systemically administering to the subject a therapeutically effective amount of a single-domain antibody (sdAb), wherein the sdAb comprises the amino acid sequence as set forth in SEQ ID NO:1, and wherein the sdAb is administered at a dose of about 10 mg/kg body weight and inhibits STAT3 activation in CD4+ T-cells, thereby reducing Th17-mediated inflammation in the subject.

2. The method of claim 1, wherein the subject is a mammal.

3. The method of claim 1, wherein the subject is a human.

4. The method of claim 1, wherein the sdAb is administered in combination with one or more anti-inflammatory or immunosuppressive agents.

5. The method of claim 1, wherein the uveitis comprises autoimmune uveitis selected from the group consisting of sympathetic ophthalmia, birdshot retinochoroidopathy, Behcet's disease, Vogt-Koyanagi-Harada disease, and ocular sarcoidosis.

6. The method of claim 1, wherein the sdAb is administered at least once daily.

7. The method of claim 1, wherein the sdAb is administered twice daily.

8. The method of claim 1, wherein administration results in an increase in electroretinogram (ERG) amplitude relative to untreated control.

9. The method of claim 8, wherein the increase is at least 10%.

10. A pharmaceutical composition comprising a single-domain antibody (sdAb) having the amino acid sequence set forth in SEQ ID NO:1 and a pharmaceutically acceptable carrier.

11. The pharmaceutical composition of claim 10, wherein the pharmaceutically acceptable carrier comprises phosphate-buffered saline (PBS), saline, or water.

12. The pharmaceutical composition of claim 10, wherein the composition is formulated as a solution or suspension.

13. The pharmaceutical composition of claim 10, wherein the composition further comprises human serum albumin.

14. The pharmaceutical composition of claim 10, wherein the composition is formulated for systemic administration.

15. The pharmaceutical composition of claim 10, wherein the composition is formulated for ocular administration.

16. A method of modulating an immune response associated with uveitis in a subject, comprising administering a single-domain antibody (sdAb) comprising the amino acid sequence of SEQ ID NO:1, wherein the sdAb inhibits STAT3 phosphorylation in CD4+ T-cells and reduces production of IL-17.

17. The method of claim 16, wherein the method reduces expansion of Th17 cells.

18. The method of claim 16, wherein the method reduces levels of one or more cytokines selected from IL-17, IFN-γ, GM-CSF, or IL-23.

19. The method of claim 16, wherein the sdAb penetrates lymphocytes and inhibits intracellular STAT3 signaling.

20. The method of claim 16, wherein treated T-cells exhibit reduced ability to induce uveitis upon transfer to a subject.


SEQUENCE LISTING (2 sequences)

SEQ ID NO: 1 - protein, 127 aa, synthetic construct
  REGION 1..127: note = Camelid
HVQLVESGGGSVQAGGSLRLSCAASGANGGRSCMGWFRQVPGKEREGVSGISTGGLITYYADSVKGRFTISQDNTKNTLYLQMNSLKPEDTAMYYCATSRFDCYRGSWFNRYMYNSWGQGTQVTVSS

SEQ ID NO: 2 - DNA, 381 bp, synthetic construct
  misc_feature 1..381: note = Camelid
  source 1..381: mol_type = other DNA
catgtgcagctggtggagtctgggggaggctcggtgcaggctggagggtctctgagactctcctgtgcagcctctggagccaacggtggtcggagctgcatgggctggttccgccaggttccagggaaggagcgcgagggggtttctggtatttcaaccggtggtcttattacatactatgccgactccgtgaagggccgattcaccatctcccaagacaacaccaagaacacgctgtatctgcaaatgaacagcctgaaacctgaggacactgccatgtactactgtgcgacgagtcggtttgactgctatagaggctcttggttcaaccgatatatgtataacagttggggccaggggacccaggtcactgtctcctca